(19) United States
(12) Patent Application Publication
(10) Pub. No.: US 2011/0238072 A1
(43) Pub. Date: Sep. 29, 2011

(54) Minimally Invasive Surgical (MIS) Technique and System for Performing an Interbody Lumbar Fusion with a Navigatable Intervertebral Disc Removal Device and Collapsible Intervertebral Device

(76) Inventor: Dwight S. Tyndall, Flossmoor, IL (US)

(21) Appl. No.: 12/732,734

(22) Filed: Mar. 26, 2010

(51) Int. Cl. A61B 17/00; A61B 17/56

(52) U.S. Cl. 606/84; 606/86 A

(57) ABSTRACT

A technique and system is provided for efficiently, effectively and safely performing an interbody lumbar fusion. The technique and system utilizes: (1) a rotary shaver that provides a shaving cannula; (2) an outflow/insertion cannula which provides a dual purpose cannula; and (3) a fusible balloon which provides a biodegradable intervertebral bag. The patient's disc is shaved and cut out with the shaver while the shavings are sucked out with the dual purpose cannula. Afterwards, the balloon is inserted at the tip of the dual purpose cannula and the balloon is filled with a grafting substance. The balloon assumes the shape of the removed disc. The grafting substance will fuse to the balloon and to the adjacent vertebrae and provide a support where the removed disc was.

### BACKGROUND OF THE INVENTION

This invention relates to spinal surgery, and more particularly, to a technique and system for performing an interbody lumbar fusion.

A herniated disc (disk), herniated nucleus pulposis, ruptured lumbar invertebral disc, slipped disc, prolapsed disc, compressed disc, degenerative disc, injured disc, or damaged disc, can cause sever back pain. One successful method of treatment involves removing the disc, also referred to as “diskectomy”, and spinal fusion, also known as “spondylodesis” or “spondylosyndesis”.

The standard way of performing a lumbar intervertebral fusion is either through an anterior approach or through a posterior approach. Either approaches involve fairly extensive dissection by which the intervertebral disc is removed and an implant, polyether ether ketone (PEEK) and/or bone, is placed into the disc space. The goal of the implanted device is to provide support for the adjacent vertebrae bodies and to ultimately fuse the adjacent vertebral bodies. Although this method can be highly effective there can be considerable postsurgical morbidity due to the extensile approach that is necessary.

It is, therefore, desirable to provide an improved technique and system, which overcomes most, if not all of the preceding disadvantages.

### BRIEF SUMMARY OF THE INVENTION

An improved minimally invasive surgical (MIS) technique and system are provided for performing an interbody lumbar fusion which is effective, efficient and safe. The improved MIS technique and system features a navigable disc removal device and an osteoconductive, osteoinductive or osteogenic, collapsible intervertebral balloon.

Among the many benefits of the improved technique and system are:
1. no need to perform an extensile dissection;
2. uses MIS technique and therefore minimizes soft tissue dissection; and
3. the disc annulus and all ligaments surrounded the disc are spared.

The patient friendly technique and system utilizes a two cannula posterior-lateral approach to the disc space. Both cannulas can be inserted just lateral to the lumbar facets within the safe zone below the exiting nerve of the corresponding disc.

One cannula can have a cutting/shaving tip which is navigatable and the other cannula can have a suction device to extract the shaved disc and for placement of the intervertebral device. The shaving cannula can also have water inflow to introduce water into the disc space to aid extraction.

On completion of disc extraction, a balloon made of a biodegradable substance such as polylatic acid or polylactide (PLA) which is a biodegradable, thermoplastic, aliphatic polyesteror, other biodegradable material can be introduced into the disc space via the extraction cannula.

The balloon preferably has specific structural characteristics:
1. not round or oval;
2. collapsible on introduction;
3. flat to concave on upper and lower surfaces when deployed; and
4. sides that are expandable in a cranial/caudal direction, medial/lateral direction, and anterior/posterior direction, which allows the device (balloon) to fully occupy the disc space.

Once the balloon is inserted it can be filled with a fusion graft substance which on insertion and pressure becomes firm and weight bearing. After the balloon is filled with a fusion graft substance, both cannulas can be removed.

In the preferred form, the MIS system comprises: a navigable intervertebral disc removal device; an intervertebral balloon; and a dual purpose cannula which has an extraction portion for extracting a shaved disc of a patient and has an insertion portion for inserting the balloon in a space where the shaved disc was extracted.

The navigable intervertebral disc removal device can comprise a shaver assembly that provides a shaving cannula and/or rotary shaver. Preferably, the navigable intervertebral disc removal device has at least one power driven blade. The illustrated navigable intervertebral disc removal device can also include a shaft connected to the blade and a set of moveable shaver discs which are spaced from the blade and connected to the shaft for accommodating axial and transverse movement of the shaft. The illustrated navigable intervertebral device can also comprise a casing with a head having a shaver access opening for the blade and an outflow irrigation opening and can have a steerability (steering) section that is spaced from the shaver access opening. The navigable intervertebral disc removal advice can also comprise a flexible steering rod. The illustrated navigable intervertebral disc removal device can further have a water inflow tube.

The preferred balloon comprises an osteoconductive, osteoinductive or osteogenic, collapsible intervertebral balloon comprising biodegradable material. The illustrated balloon has a contour replicating and complementing a patient's disc.

A more detailed explanation of the invention is provided in the following detailed descriptions and appended claims taken in conjunction with the accompanying drawings.

### BRIEF DESCRIPTION OF THE DRAWINGS

FIG. 1 is a top profile perspective view of a navigable intervertebral disc removal device of a minimally invasive surgical (MIS) system for performing an interbody lumbar fusion in accordance with principles of the present invention.

FIG. 2 is a side profile perspective view of the navigable intervertebral disc removal device.

FIG. 3 is a cross sectional side view of an osteoconductive, osteoinductive or osteogenic, collapsible intervertebral balloon providing an intervertebral bag of the MIS system for performing an interbody lumbar fusion in accordance with principles of the present invention.

FIG. 4 is a back profile perspective view of the navigable intervertebral disc removal device being inserted into an intervertebral disc of a patient.

FIG. 5 is a posterior profile perspective view of a spinal segment of the patient.

FIG. 6 is a cross sectional top profile view of the spinal segment of the patient.

FIG. 7 is a top profile view of the navigable intervertebral disc removal device and a dual action cannula that provides an outflow/insertion cannula of the MIS technique and system for performing an interbody lumbar fusion in the spinal segment of the patient in accordance with principles of the present invention and illustrating in dotted line movement of the navigable intervertebral disc removal device for shaving and cutting the patient's disc into pieces.

FIG. 8 is a cross sectional top profile view of the osteoconductive, osteoinductive or osteogenic, collapsible intervertebral balloon inserted by the dual action cannula and an insertion rod into the disc space after the patient's disc has been removed.

FIG. 9 is a cross sectional side profile view of the osteoconductive, osteoinductive or osteogenic, collapsible intervertebral balloon inserted by the dual action cannula and insertion rod into the disc space after the patient's disc has been removed.

FIG. 10 is a cross sectional side profile view of the osteoconductive, osteoinductive or osteogenic, collapsible intervertebral balloon inserted by the dual action cannula and insertion rod in the disc space and being filled by a grafting substance to form a fusion support (fusion implant) to securely connect and fuse adjacent vertebrae bodies.

FIG. 11 is a cross sectional side profile view of the fusion support in the disc space for fusing the fusion material to the osteoconductive, osteoinductive or osteogenic, collapsible intervertebral balloon and adjacent vertebrae bodies after the dual action cannula and insertion rod have been removed.

FIG. 12 is a perspective view of a shaver with a shaver blade, shaft and stackable discs.

### DETAILED DESCRIPTION OF THE INVENTION

The following is a detailed description and explanation of the preferred embodiments of the invention and best modes for practicing the invention.

Referring to the drawings, a minimally invasive surgical (MIS) system 100 is provided for performing interbody lumbar fusion. The MIS system has: a navigable intervertebral disc removal device 102 for removing a patient's disc 104; and a device providing an osteoconductive, osteoinductive or osteogenic, collapsible intervertebral balloon 106; and a dual action cannula 108. The term “osteoconductive” as used in this application means that the balloon assists in replacing the disc space 110 previously occupied by the patient's disc with a fusion support (fusion implant) so as to allow bones to form between and fuse to adjacent vertebrae bodies of the patient. The term “osteoinductive” as used in this application means that the balloon assists in replacing the disc space 110 previously occupied by the patient's disc with a fusion support (fusion implant) so as to induce bones to form between and fuse to adjacent vertebrae bodies of the patient. The term “osteoconductive” as used in this application means that the balloon assists in replacing the disc space 110 previously occupied by the patient's disc with a fusion support (fusion implant) so as to form bones between and fuse to adjacent vertebrae bodies of the patient.

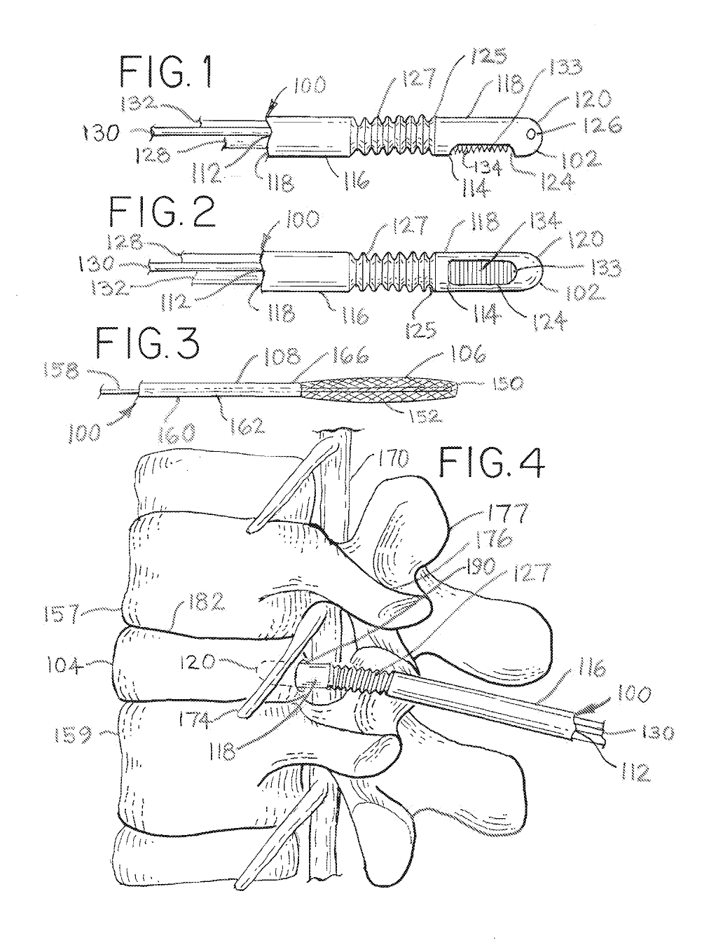

The navigable intervertebral disc removal device can comprise a shaver assembly 112 (FIGS. 1 and 2) that provides a shaver cannula 114. The navigable intervertebral disc removal device can have an outer casing 116 with an interior 118 and an elongated head 120 with a rounded convex tip 122. The head can have a shaver access opening 124, such as a 0.5 cm opening, and an outflow irrigation opening 126 or hole which provide a water outlet for injecting and spraying sterile water. The navigable intervertebral disc removal device can be constructed of a medical grade material, such as titanium, stainless steel or other metal or impact resistant plastic. The casing can have a steerability (steering) section with an accordion-shaped or style connection 127 that is spaced from the shaver access opening and allows steering, maneuvering and navigation of the navigable intervertebral device. The navigable intervertebral disc removal device (shaver assembly) can also include: a shaft 128 that extends into the casing, a flexible steering rod 130 that extends into the casing, and a water inlet tube 132 that extends into the casing and communicates with the outflow irrigation opening of the head. The water inflow tube can be made of medical grade material, such as plastic or rubber, and can be connected to a sterile water source such a three liter plastic bag of water. The sterile water can be pumped by a water pump or can flow by gravity flow.

The navigable intervertebral disc removal device can have a shaver 133 at least one power driven circular blade 134, such as a spiral or helical blade or a set, series, or array of circular blades, such as a 0.5 cm blade, that is accessible and communicates with the shaver access opening and is positioned within the interior of the casing for shaving, cutting and removing the patient's disc. The power driven circular blade can be a pneumatically operated rotary and/or oscillating circular blade or an electrically powered rotary and/or oscillating circular blade. A pneumatic pump or electrical motor can be mounted on or adjacent a wall in the hospital where the procedure (technique) is being performed to power the blade. The power drive circular blade can be positioned at or near a tip of the shaft.

The navigable intervertebral disc removal device can also have an array, series or set of stackable moveable shaver discs 136 (FIG. 12) which are spaced from the blade of the shaver and are connected to the shaft for accommodating longitudinal and lateral movement and motion of the shaft. The shaver discs can be constructed of metal or impact resistant plastic. In the illustrated embodiment, the shaver discs comprise complementary rotatable shaver discs. Each shaver disc can have a convex forward portion 138 and a rearward concave, planar or flat portion 140. At least some of the concave portions can have a cavity 142 which provides a nipple-receiving opening 144. At least some of the convex portions can have an axial protuberance 146 which provides a nipple 148 for engaging and is aligned with the cavity of an adjacent rearward portion. Desirably, the shaver discs are moveable in a transverse direction.

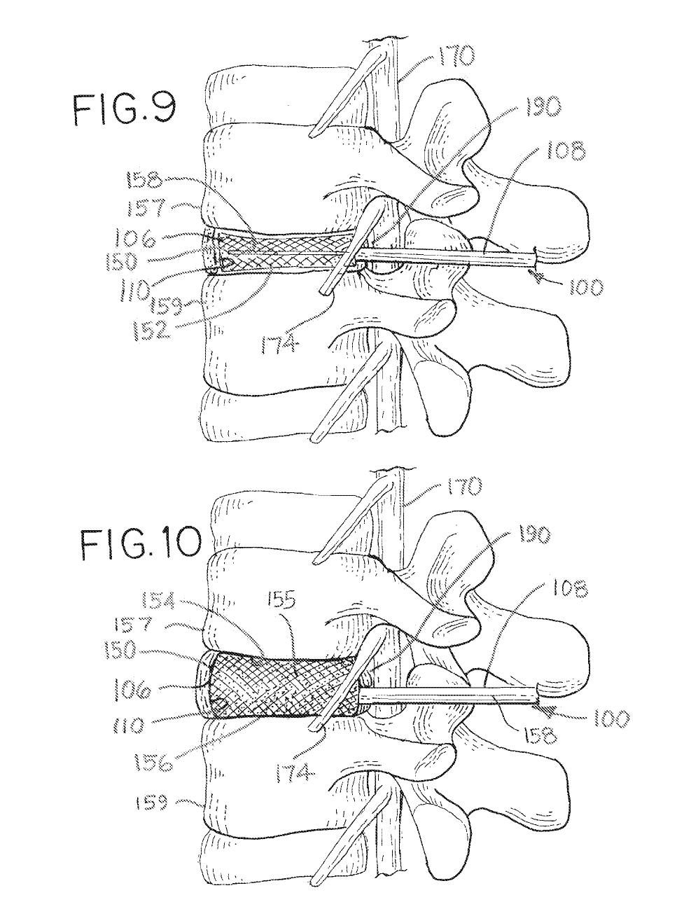

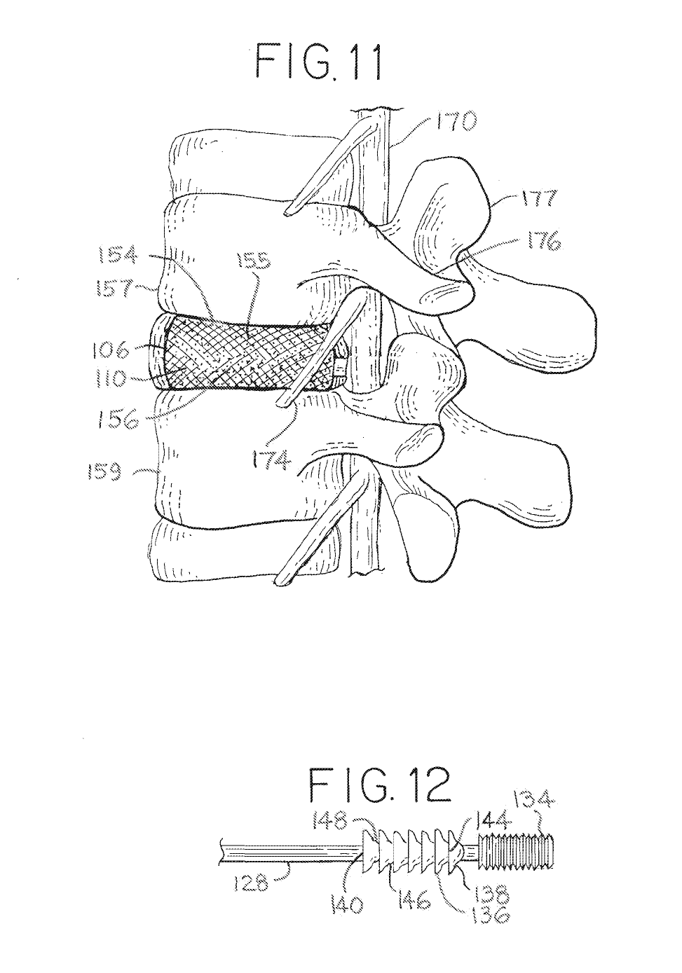

The osteoconductive, osteoinductive or osteogenic, collapsible intervertebral balloon preferably comprises an intervertebral bag 150 (FIG. 3) with an expandable contour 152 for replicating and complementing a patient's disc. Before being expanded, the balloon is in a normally collapsed storage position in an extended and deployed form which is generally flat or slightly concave. The intervertebral bag can be expandable and/or inflatable to an expanded position in a cranial and caudal direction for occupying a disc space after the patient's disc has been removed, so that the annulus is spared. The intervertebral bag can have an interior 154 (FIG. 10) to receive a grafting material 156 which provides a graft substance, such as Grafton brand grating material that is available from Osteotech, Inc. The grafting material can fuse and provide a fusion support 155 (fusion implant) (FIG. 11) in the disc space for securely connecting and fusing adjacent vertebrae bodies 157 and 159 of the patient. Desirably, the intervertebral balloon comprises a medical grade biodegradable or bio-absorbable fusible material, such as polylatic acid or polylactide (PLA) which is a biodegradable thermoplastic, aliphatic polyesteror, or other plastic or paper, which can be absorbed and fused with the fusion support. A grafting material-engageable insertion rod 158 (FIGS. 8 and 9) can be provided for inserting and pushing the graft substance into the interior of the intervertebral bag. The insertion rod can also give rigidity to the balloon. The insertion rod can be made of a medical grade material, such as titanium, stainless steel, or other metal or impact-resistance plastic.

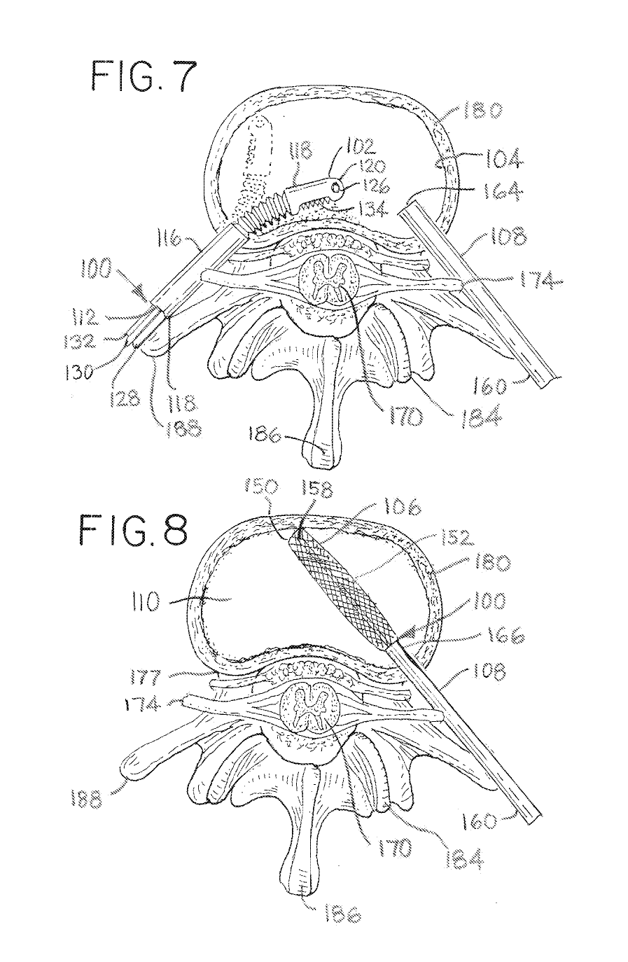

The dual action cannula can comprise a tube 160 (FIG. 3) that provides an outflow/insertion cannula 162. The dual action cannula can have an extraction section 164 (FIG. 7) which provides a suction device and outflow inlet portion for extracting and discharging the removed patient's disc. The dual action cannula can also have an insertion portion 166 (FIG. 8) for inserting the intervertebral balloon in the disc space after the patient's disc has been removed. The tubular dual action cannula can be fabricated of a medical grade material, such as titanium, stainless steel or other metal or impact resistant plastic.

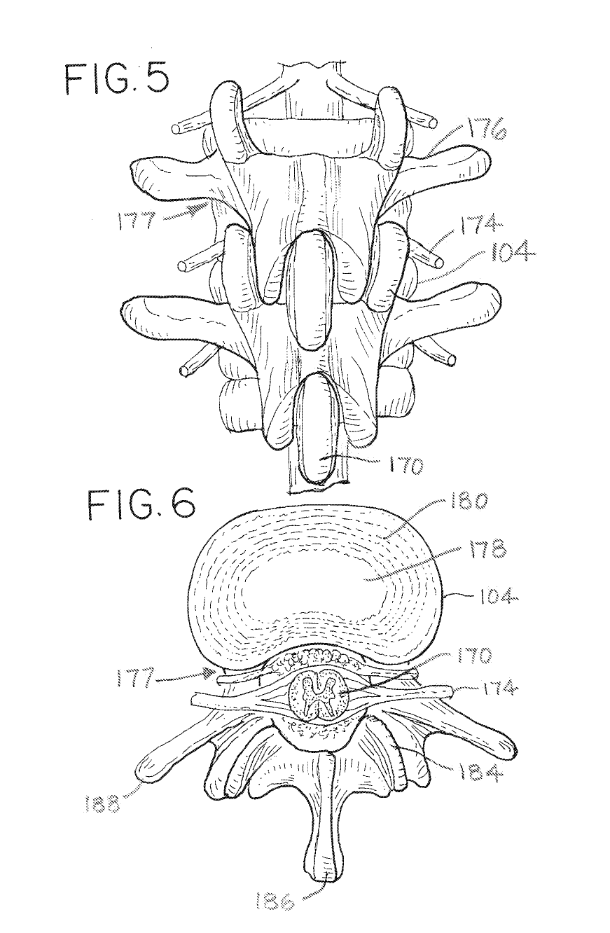

The back of the patient has a spinal cord 170 (FIGS. 4-10) with nerve roots 174 and pedicels 176. The patent also has spinal segments 177 with a nucleus pulposus 178 (FIG. 6), annulus fibrosus 180, hyaline cartilage 182 (FIG. 4), superior articular facet 184 (FIG. 6), spinous process 186, and transverse process 188.

The MIS system is particularly useful in a minimally invasive surgical (MIS) process which provides a technique and/or procedure for performing interbody lumbar fusion. The patient is preferably administered a general anesthetic before the MIS process. The process can include inserting and navigating a navigable intervertebral disc removal device in a nucleus pulposis of a patient posteriorly and laterally of a lumbar facet and in proximity to a patient's disc in a safe zone 190 below an exiting nerve of the patient's disc. The dual action cannula can be inserted and positioned in the nucleolus pulposis posteriorly and laterally of another lumbar facet and in proximity to the patient's disc in the safe zone. The dual action cannula can be inserted before or after the navigable intervertebral disc removal device or simultaneously therewith.

The MIS process, technique and procedure preferably include shaving and substantially removing the patient's disc with the intervertebral disc removal device and extracting and discharging the removed patient's disc with the dual action cannula. In the illustrated embodiment, the patient's disc is shaved with at least one rotatable cutting blade of the navigable intervertebral disc removal device and the cutting blade is pneumatically or electronically driven and rotated. Preferably, sterile water is injected through the water inlet tube into the shaving cannula about the blade during shaving and cutting to cool the blade and assist in extracting and discharging the disc shavings (cuttings). Advantageously, the cutting blade can be moved longitudinally and laterally with stackable shaver discs.

The osteoconductive, osteoinductive or osteogenic, collapsible intervertebral balloon can be inserted with the dual action cannula into a disc space previously occupied by the patient's disc. A grafting substance can then be dispensed into the balloon in the disc space. Advantageously, the grafting substance and balloon are fused in the disc space adjacent vertebrae bodies of the patient to form a fusion support (fusion implant) there between. Preferably, the balloon comprises a biodegradable material and is absorbed in the fusion support.

In the preferred process and system, the balloon comprises an intervertebral bag which is expanded in a cranial and caudal direction to fill the disc space and provide a contour that replicates the shape of the patient's disc. After the grafting substance is dispensed and inserted into the intervertebral bag, the grating substance is compacted and compressed, such as with the grafting material-engageable insertion rod. The navigable intervertebral disc removal device and/or dual action cannula can be removed after the grafting substance is dispensed into the intervertebral balloon.

Among the many advantages of the MIS Technique and System for Performing an Interbody Lumbar Fusion are:
1. No need to perform an extensile dissection.
2. Uses MIS technique therefore minimizes soft tissue dissection.
3. The disc annulus and all ligaments surrounded the disc are spared.
4. The ability to remove the patient's disc with a navigatable shaver cannula.
5. The fusion balloon is not round but rather replicates the shape of the patient's disc and spread the forces out along the entire end place of the vertebral body.
6. The balloon is made of a bio-absorbable substance which allows the balloon to incorporate and become part of the fusion and fusion support.
7. Superior MIS technique and system.
8. Superb capabilities for removing a patient's disc.
9. Outstanding performance.
10. Reliable.
11. Safe.
12. Light weight.
13. Portable.
14. Patient friendly.
15. Easy to use.
16. Durable
17. Economical.
18. Attractive.
19. Efficient.
20. Effective.

Although embodiments of the invention have been shown and described, it is to be understood that various modifications, substitutions, and rearrangements of parts, components, equipment, and/or process (method) steps, as well as other uses of the MIS system and techique, can be made by those skilled in the art without departing from the novel spirit and scope of this invention.

What is claimed is:

1. A minimally invasive surgical (MIS) system for performing interbody lumbar fusion, comprising:
a navigable intervertebral disc removal device;
an intervertebral balloon; and
a dual purpose cannula having an extraction portion for extracting a shaved disc of a patient and having an insertion portion for inserting said balloon in a space where the shaved disc was extracted.

2. A MIS system in accordance with claim 1 wherein said navigable intervertebral disc removal device comprises a shaver assembly.

3. A MIS system in accordance with claim 2 wherein said shaver assembly provides a shaving cannula.

4. A MIS system in accordance with claim 1 wherein said navigable intervertebral disc removal device has at least one power driven blade.

5. A MIS system in accordance with claim 4 including:
a shaft connected to said blade; and
a set of moveable shaver discs spaced from said blade and connected to said shaft for accommodating axial and transverse movement of said shaft.

6. A MIS system in accordance with claim 4 wherein said navigable intervertebral device comprises a casing with a head having a shaver access opening for said blade and defining an outflow irrigation opening, and said casing having a steerability section spaced from said shaver access opening.

7. A MIS system in accordance with claim 1 wherein said navigable intervertebral disc removal advice comprises a flexible steering rod.

8. A MIS system in accordance with claim 1 wherein said navigable intervertebral disc removal device comprises a water inflow tube.

9. A MIS system in accordance with claim 1 wherein said balloon comprises an osteoconductive, osteoinductive or osteogenic, collapsible intervertebral balloon comprising biodegradable material.

10. A MIS system in accordance with claim 1 wherein said balloon has a contour replicating and complementing a patient's disc.

11. A minimally invasive surgical (MIS) process providing a technique for performing interbody lumbar fusion, comprising the steps of:
inserting and navigating a navigable intervertebral disc removal device in a nucleus pulposis of a patient posteriorly and laterally of a lumbar facet and in proximity to a patient's disc in a safe zone below an exiting nerve of the patient's disc;
inserting and positioning a dual action cannula in the nucleolus pulposis posteriorly and laterally of another lumbar facet and in proximity to the patient's disc in the safe zone;
shaving and substantially removing the patient's disc with the intervertebral disc removal device;
extracting and discharging the removed patient's disc with the dual action cannula;
inserting an osteoconductive, osteoinductive or osteogenic, collapsible intervertebral balloon with the dual action cannula into a disc space previously occupied by the patient's disc;
dispensing a grafting substance into the balloon in the disc space; and
fusing the grafting substance and balloon in the disc space adjacent vertebrae bodies to form a fusion support there between.

12. A MIS process in accordance with claim 11 wherein:
the patient's disc is shaved with at least one rotatable cutting blade of the navigable intervertebral disc removal device; and
the navigable intervertebral disc removal device comprises a shaver assembly providing a shaving cannula.

13. A MIS process in accordance with claim 12 wherein:
the cutting blade is pneumatically or electronically driven and rotated; and
water is injected into the shaving cannula about the blade.

14. A MIS process in accordance with claim 11 wherein:
the cutting blade is moved longitudinally and laterally with stackable shaver discs; and
the dual action cannula is inserted before or after the navigable intervertebral disc removal device.

15. A MIS process in accordance with claim 11 wherein:
the balloon comprises a biodegradable material; and
the balloon is absorbed in the fusion support.

16. A MIS process in accordance with claim 1 wherein:
the balloon comprises an intervertebral bag;
expanding the bag in a cranial and caudal direction to fill the disc space and provide a contour that replicates the shape of the patient's disc;
compacting and compressing the grafting substance in the bag;
removing the dual action cannula after the grafting substance is dispensed into the balloon; and
removing the navigable intervertebral disc removal device after the grafting substance is dispensed into the balloon.

17. A minimally invasive surgical (MIS) system for performing interbody lumbar fusion, comprising:
a navigable intervertebral disc removal device for removing a patient's disc comprising a shaver assembly providing a shaver cannula, with an outer casing having an interior and a head with a shaver access opening and/an outflow irrigation opening, said casing having a steerability section with an accordion connection spaced from said shaver access opening, a shaft extending into said casing, at least one power driven circular blade accessible and communicating with said shaver access opening and positioned within the interior of said casing for shaving the patient's disc, said blade positioned in proximity to a tip of said shaft, an array of stackable moveable shaver discs spaced from said blade and connected to said shaft for accommodating longitudinal and lateral movement of said shaft, a water inlet tube extending into said casing, and a flexible steering rod extending into said casing;
an osteoconductive, osteoinductive or osteogenic, collapsible intervertebral balloon comprising an intervertebral bag with an expendable contour for replicating and complementing a patient's disc, said bag being expandable in a cranial and caudal direction for occupying a disc space after the patient's disc has been removed, said bag having an interior for receiving a grafting material providing a graft substance for fusing and providing a fusion support for adjacent vertebrae bodies in the disc space, said balloon comprising medical grade biodegradable fusible material for being absorbed and fused with the fusion support; and
a dual action cannula having an extraction section providing a suction device for extracting and discharging the removed patient's disc and having an insertion portion for inserting said balloon in the disc space after the patient's disc has been removed.

18. A MIS system in accordance with claim 17 wherein said shaver discs comprise complementary rotatable shaver discs, each shaver disc having a convex forward portion and a rearward portion, at least some of said concave portions having a cavity providing a nipple-receiving opening, at least some of said convex portions having a protuberance providing a nipple for engaging and aligned with the cavity of an adjacent rearward portion, and said discs being moveable in a transverse direction.

19. A MIS system in accordance with claim 18 including a grafting material-engageable rod for inserting the grafting substance into the interior of said intervertebral bag.

20. A MIS system in accordance with claim 18 wherein said power driven circular blade is selected from the group consisting of a pneumatically operated rotary and/or oscillating circular blade and an electrically powered rotary and/or oscillating circular blade.
